(19) United States
(12) Patent Application Publication
(10) Pub. No.: US 2004/0167150 A1
(43) Pub. Date: Aug. 26, 2004

(54) Treatment methods

(76) Inventors: Alan G. Harris, New York, NY (US); Paul T. Medeiros, Easton, PA (US)

Correspondence Address: SCHERING-PLOUGH CORPORATION, PATENT DEPARTMENT (K-6-1, 1990), 2000 GALLOPING HILL ROAD, KENILWORTH, NJ, 07033-0530, US

(21) Appl. No.: 10/791,477

(22) Filed: Mar. 2, 2004

Related U.S. Application Data
(63) Continuation of application No. 10/012,920, filed on Oct. 30, 2001.
(60) Provisional application No. 60/244,226, filed on Oct. 30, 2000.

(51) Int. Cl. A61K 31/473; A61K 31/47

(52) U.S. Cl. 514/290; 514/311

(57) ABSTRACT

Methods of treating and/or preventing a cardiovascular disease in a human suffering from an allergic and/or inflammatory condition or cardiovascular disease by administrating an effective amount of loratadine, in combination with an effective amount of montelukast, for such treating and/or preventing are disclosed.

### BACKGROUND OF THE INVENTION

Atherosclerosis is a major cause of cardiovascular morbidity and mortality, primarily myocardial infarction and stroke. Multiple risk factors, both inherited and acquired, determine the development and severity of atherosclerotic plaque formation. Medical management of atherosclerosis has targeted modifiable risk factors such as cigarette smoking, dyslipidemia, hypertension, diabetes mellitus and obesity. Pharmacological therapies have been directed against all of these traditional risk factors, but disease control has remained sub-optimal. With the advent of new genetic and molecular research, the complex pathophysiology of atheroma formation is now better understood, and new risk factors have been identified.

Current consensus states that atherosclerotic plaque formation is primarily an intravascular inflammatory process. Ross, R., Am. Heart J., 1999; 138: S419-S420. High levels of low-density lipoprotein (LDL) and other factors can interact with cellular and humoral immune constituents to cause lipid deposition beneath the vascular endothelium. This inflammatory immune response is propagated by continued dyslipidemia, and exacerbated by hypertension, insulin resistance, smoking and other conventional and novel risk factors. Plaque growth and eventual rupture is mediated locally by inflammatory cells, cytokines and other chemical mediators. The focus of medical research has switched to down-regulating this inflammatory process by reducing causative cells and cytokines.

Key inflammatory cells such as mast cells and eosinophils have been discovered in normal and atherosclerotic hearts and blood vessels. Mast cells are present in cardiac muscle, more specifically in the blood vessel wall (intima and adventitia), as well in human atherosclerotic blood vessel wall, preferentially at the important “shoulder” region of the plaque. Activated mast cells increase in coronary arterial atheroma plaque. Cardiac mast cells contain histamine, tryptase, and chymase. Chymase can convert Angiotensin I to Angiotensin II, which may increase cardiovascular risk by raising blood pressure. Angiotensin II may also play a role in the proliferation of smooth muscle cells that helps form atherosclerotic plaques. Chymase may also cleave bound LDL, thereby freeing it to be incorporated into atheroma by macrophages. Mast cell numbers are increased in the ischemic heart.

An important group of inflammatory mediators are the cysteinyl-leukotrienes (LT's), LTC4, LTD4 and LTE4, which are produced from arachidonic acid by many cells including endothelium, mast cells and eosinophils. LT's modulate vascular tone and cardiac contractility in addition to inducing bronchoconstriction and mucus secretion. Some LT's are also chemotactic for inflammatory cells. Perivascular mast cells, platelets and vascular smooth muscle produce LT's normally, while elevated cysteinyl-LT levels are seen in patients with myocardial infarction and unstable angina. Leukotriene receptor antagonists, such as montelukast, have been shown to antagonize the effects of cysteinyl-leukotrienes, particularly LTD4, in diseases such as asthma and allergic rhinitis.

Systemic inflammatory conditions like rheumatoid arthritis (RA), systemic lupus erythematosus (SLE) and others are associated with elevated levels of local/circulating inflammatory cells and cytokines. The risk of myocardial infarction in female patients with SLE is 50 times that of age-matched controls, Manzi, S., et al., Am. J. Epidemiol., 1997; 145: 408-415, and RA patients are twice as likely to die from cardiovascular disease as normal individuals. Prior, O., et al., Br. J. Rheumatol., 1984; 23: 92-99. It has been postulated that shared inflammatory mediators due to RA or SLE circulate systemically and increase the rate of atheroma formation. Elevated systemic inflammatory markers like immunoglobulin E (IgE), C-reactive peptide, interleukin (IL-6) and soluble intercellular adhesion molecule-1 (sICAM-1) are associated with increased risk of serious cardiovascular morbidity. Van Lente, F., Clinica Chimica Acta, 2000; 293: 31-52.

Allergy, seasonal allergic rhinitis (SAR) and perennial allergic rhinitis (PAR), allergic asthma, atopic dermatitis/eczema, and chronic idiopathic urticaria (CIU) are systemic inflammatory disorders. Mast cells, basophils, eosinophils and T-lymphocytes produce pro-inflammatory mediators like histamine, LT's, cytokines (IL-4, IL-5, IL-13), chemokines (IL-8, RANTES) and adhesion molecules (ICAM-1, P-selectin) following activation of IgE by allergens. However, many of these mediators are also implicated in atherosclerotic inflammation and cardiovascular disease. George, J., et al., Circ. Res., 2000; 86: 1203-1210, reported that IL-4 plays a requisite role in the development of early inflammatory lipid accumulation in an animal model of atherosclerosis. They suggested that IL-4 represents a target for immunological modulation of atherosclerotic lesions. Labarrere, C. A., et al., Circulation, 2000; 102: 1549-1555, and Ridker, P. M., et al., Lancet, 1998; 351: 88-92 reported that elevated sICAM-1 increased the risk of serious cardiac disease in heart transplant and healthy patients, respectively. Mendall, M. A., et al., Heart, 1997; 78: 273-277), found that cardiovascular risk and ECG abnormalities were correlated with elevated serum levels of the cytokines TNF-α and IL-6.

Epidemiological evidence has also linked allergic inflammation with cardiovascular pathology. For example, Kovanen, P. T., et al., Archives of Internal Medicine, 1998, Vol. 158, pp. 1434-1439, disclose that elevated levels IgE are associated with myocardial infarction and cardiac death in men with dyslipidemia. Kockmaz, M. E., et al., International Journal of Cardiology, 1991, Vol. 31, pp. 199-204 disclose that serum IgE levels were significantly higher in patients with unstable angina and acute myocardial infarction compared to patients with stable angina pectoris and normal humans. Criqui, M. H., et al., The American Journal of Medicine, 1987, Vol. 82, pp. 964-968, disclose a possible link between allergic disease and cardiovascular disease in men, but not in women. Furthermore, it has been reported that eosinophilia is an additional risk factor for death from cardiovascular diseases including ischemic heart disease and cerebrovascular disease. Hospes, et al., American Journal of Epidemiology, 1999; Vol. 150, (No. 5), pp. 482-491, (1999).

In summary, atherosclerosis is an inflammatory disease that is linked to other systemic inflammatory conditions like allergy by shared cells (mast cells, eosinophils) and mediators (LT's, cytokines, adhesion molecules). Patients suffering from systemic inflammation have increased risk of cardiovascular morbidity and mortality. By pharmacologically lowering the activity of and secretion by mast cells and other immunological cells in accordance with the methods of the present invention, we can lower the risk and/or severity of cardiovascular disease.

### SUMMARY OF THE INVENTION

We have discovered a safe and effective therapy for a human at risk of or suffering from a cardiovascular disease by administering an effective amount of loratadine in combination with an effective amount of montelukast for a time sufficient to reduce the risk or prevent the occurrence of a cardiovascular disease.

Thus, the present invention provides a method of treating and/or preventing a cardiovascular disease in a human suffering from an allergic and/or inflammatory condition which comprises administering to such human in need of such treating and/or preventing an effective amount of loratadine, or a pharmaceutically acceptable salt thereof, in combination with an effective amount of montelukast, or a pharmaceutically acceptable salt thereof.

The present invention also provides a method of treating and/or preventing a cardiovascular disease in a human in need of such treating and/or preventing which comprises administering to such human in need of such treating and/or preventing an effective amount of loratadine, or a pharmaceutically acceptable salt thereof, in combination with an effective amount of montelukast, or a pharmaceutically acceptable salt thereof.

The present invention also provides a method of treating and/or preventing a cardiovascular disease in a human suffering from seasonal or perennial allergic rhinitis which comprises administering to such human in need of such treating and/or preventing an effective amount of loratadine, or a pharmaceutically acceptable salt thereof, in combination with an effective amount of montelukast, or a pharmaceutically acceptable salt thereof.

The present invention also provides a method of treating and/or preventing a cardiovascular disease in a human suffering from atopic dermatitis or urticaria which comprises administering to such human in need of such treating and/or preventing an effective amount of loratadine, or a pharmaceutically acceptable salt thereof, in combination with an effective amount of montelukast, or a pharmaceutically acceptable salt thereof.

### DETAILED DESCRIPTION OF INVENTION

We have found that immune cells and mediators/messengers which are responsible for disease expression in allergy also increase the risk of and/or are implicated in the development of cardiovascular disease. These shared inflammatory cells include mast cells, eosinophils and neutrophils, while shared immunological mediators/messengers include leukotrienes, cytokines (e.g., IL-4, IL-6 and TNF-α), and adhesion molecules.

Loratadine, a potent H1-histamine receptor antagonist has been shown to have inhibitory effects on systemic inflammatory cells and mediators. Bewtra, et al. J. Alllergy Clin. Immunol., 1993; 91: 639, discovered that loratadine inhibited the activity and chemotaxis of eosinophils, while Paubert-Braquet M., et al., J. Allergy Clin. Immunol., 1994; 93: 257 (Abstract 567), reported that loratadine reduced the activation of monocytes as measured by superoxide anion production. Loratadine has been found to inhibit the release of mast cell histamine and the cysteinyl leukotriene, LTC4, from allergic immune cells in vitro. Genovese, A., et al., Clin. Exp. Allergy, 1997; 27: 559-567. Molet, S., et al., Clin. Exp. Allergy, 1997; 27: 1167-1174, discovered that loratadine inhibited the inflammatory cytokine IL-6, while Vignola, A. M., et al., Allergy, 1995; 50: 200-203, found that loratadine reduced adhesion molecule ICAM-1 expression.

WO 97/28797 reportedly discloses a method of treating asthma, allergy, and inflammation by administering loratadine with a leukotriene inhibitor.

We have found that administering therapeutically effective amounts of loratadine in combination with montelukast is useful in treating and/or preventing cardiovascular disease in patients having inflammatory conditions, especially those patients suffering from an allergic and/or inflammatory condition. In a preferred embodiment of the present invention, loratadine in combination with montelukast is administered to those patients, such as type 2 diabetic patients afflicted with minimal persistent allergic inflammation, to prevent or lower the risk of developing cardiovascular disease. In another embodiment, loratadine in combination with montelukast is administered to those patients, such as type 2 diabetic patients afflicted atherosclerotic disease, to prevent or lower the risk of developing cardiovascular disease.

U.S. Pat. No. 4,282,233 discloses methods of making loratadine, pharmaceutical compositions containing it and methods of using loratadine and pharmaceutical compositions containing it to effect an anti-allergic response in mammals. Loratadine is available from Schering-Plough Corporation, Kenilworth, N.J. under the Claritin™ Tradename.

The amount of loratadine effective for use in the present invention will vary with the age, sex, body weight, severity of the allergic and inflammatory condition and the response of the patient. Typically, the amount of loratadine effective for treating or preventing such allergic and inflammatory conditions is in the range of about 1 mg/day to about 45 mg/day, preferably about 2.5 mg/day to about 20 mg/day, or about 5.0 mg/day to about 15 mg/day, or about 5.0 mg/day to about 10 mg/day, and most preferably about 10.0 mg/day in single or divided doses, or a single dose of 10.0 mg/day.

Montelukast is a leukotriene D4 antagonist capable of antagonizing the receptors for the cysteinyl leukotrienes. The technical name of montelukast is [R-(E)]-1-[[[1-[3-[2-(7-chloro-2-quinolinyl)ethenyl]phenyl]-3-[2-(1-hydroxy-1-methylethyl)phenyl]propyl]thio]methyl]-cyclopropaneacetic acid. This compound is described in EP 480,717. A preferred pharmaceutically acceptable salt of montelukast is the monosodium salt, also known as montelukast sodium. The amount of montelukast which can be employed in a unit dosage form of the present invention can range from about 1 to 100 milligrams, also from about 5 to about 20 milligrams, preferably about 10 milligrams.

The pharmaceutical compositions of the present invention can be administered depending upon the patient's age, sex, weight and severity of the condition being treated. Generally, the human oral dosage form containing loratadine, or a pharmaceutically acceptable salt thereof, and montelukast, or a pharmaceutically acceptable salt thereof, can be administered 1 or 2 times per day, preferably once a day. Preferably the pharmaceutical composition is designed for oral administration. Most preferably, loratadine and montelukast are admixed in a single unit dose designed for oral administration.

The term “in combination with” as used herein means that the antihistamines my be administered contemporaneously or sequentially with montelukast as separate pharmaceutical compositions or together in one pharmaceutical composition.

Pharmaceutical compositions of the present invention may be formulated by combining loratadine, or an equivalent amount of a pharmaceutically acceptable salt thereof, with montelukast, or an equivalent amount of a pharmaceutically acceptable salt thereof, with a suitable, inert, pharmaceutically acceptable carrier or diluent that may be either solid or liquid.

Solid form preparations include powders, tablets, rapidly disintegrating tablets, dispersible granules, capsules, cachets and suppositories. The powders and tablets may be comprised of from about 5 to about 95 percent active ingredients. Suitable solid carriers are known in the art, e.g. magnesium carbonate, magnesium stearate, talc, sugar or lactose. Tablets, powders, cachets and capsules can be used as solid dosage forms suitable for oral administration. Examples of pharmaceutically acceptable carriers and methods of manufacture for various compositions may be found in A. Gennaro (ed.), Remington's Pharmaceutical Sciences, 18th Edition, (1990), Mack Publishing Co., Easton, Pa.

Liquid form preparations include solutions, suspensions, syrups and emulsions. As an example may be mentioned water or water-propylene glycol solutions for parenteral injection. Solid form preparations may be converted into liquid preparations shortly before use for either oral or administration. Parenteral forms to be injected intravenously, intramuscularly or subcutaneously are usually in the form of sterile solutions and may contain tonicity agents (salts or glucose), and buffers. Opacifiers may be included in oral solutions, suspensions and emulsions. Liquid form preparations may also include solutions for intranasal administration.

Aerosol preparations suitable for inhalation may include solutions and solids in powder form, which may be in combination with a pharmaceutically acceptable carrier, such as an inert compressed gas, e.g., nitrogen.

Also included are solid form preparations which are intended to be converted, shortly before use, to liquid form preparations for either oral or parenteral administration. Such liquid forms include solutions, suspensions and emulsions.

The compounds of the invention may also be deliverable transdermally. The transdermal compositions can take the form of creams, lotions, aerosols and/or emulsions and can be included in a transdermal patch of the matrix or reservoir type as are conventional in the art for this purpose.

Preferably, the pharmaceutical preparation is in a unit dosage form. In such form, the preparation is subdivided into suitably sized unit doses containing appropriate quantities of the active component, e.g., an effective amount to achieve the desired purpose.

The pharmaceutical compositions of loratadine and montelukast can be adapted for any mode of administration e.g., for oral, parenteral, e.g., subcutaneous (‘SC”), intramuscular (“IM”), intravenous (“IV)” and intraperitoneal (“IP”), topical or vaginal administration or by inhalation (orally or intranasally). Preferably loratadine and montelukast are administered orally.

An eosinophil is a type of white blood cell which normally represents about 8% of the total white blood cell population in the circulating blood. Eosinophilia is the formation and the accumulation of eosinophils above the normal level of about 350 copies per μL of peripheral blood. The development of eosinophilia has features of an immune response and occurs in diseases, including seasonal and perennial allergic rhinitis, asthma, urticaria, eczema, atopic dermatitis, parasite infections, drug reactions and connective tissue disease, such as rheumatoid arthritis and scleroderma. Infiltration of the airways by eosinophils is an especially important factor in the development of airway inflammation that contributes to the pathophysiology of bronchial asthma and allergic rhinitis.

The term “patients in need of such treating and/or preventing” as used herein means those patients at risk of cardiovascular disease as identified by traditional coronary risk factors enumerated above, as well as those having an allergic and/or inflammatory condition, elevated serum levels of eosinophils and/or immunoglobulin levels, e.g., IgA, IgE, IgG and IgM compared to those found in normal subjects. Serum immunoglobulin and eosinophil levels may be measured by standard commercially available quantitative immunoturbidimetry techniques, e.g., an automated clinical chemistry analyzer (KoneSpecific R, Kone Instruments, Espoo, Finland). IgE serum levels may also be measured using an automated microparticle enzyme immunoassay such as IMx available from Abbott Diagnostics, U.S.A. and serum IgG levels may be also assessed by nephelometry (Behring, Germany).

The phrase “an allergic and/or inflammatory condition” as used herein means those allergic and/or inflammatory conditions and symptoms found on the skin and in the airway passages from the nose to the lungs. Typical allergic and/or inflammatory conditions of the skin and upper and lower airway passages include seasonal and perennial allergic rhinitis, non-allergic rhinitis, asthma including allergic and non-allergic asthma, sinusitis, colds, dermatitis, especially allergic and atopic dermatitis and urticaria. Inhibition of eosinophil infiltration and/or function may be implicated in the reduction of airway inflammation and thus alleviate development of bronchial asthma and allergic rhinitis.

Typically suitable eosinophilia-related and immunoglobin-related allergic and/or inflammatory conditions of the skin or the upper and lower airway passages include, but are not limited to, allergic asthma, seasonal allergic rhinitis, perennial allergic rhinitis, atopic dermatitis, and chronic obstructive lung disease.

The term “cardiovascular disease” means diseases related to the heart and the blood vessels or the circulation, such as atherosclerosis, ischemic heart disease or cerebrovascular disease such as coronary artery disease including angina pectoris and myocardial infarction, stroke, vascular heart disease and peripheral vascular disorders such as peripheral arterial disease and occlusive arterial diseases.

The present invention also contemplates use of loratadine and montelukast in combination with one of more of the therapies useful for lowering serum cholesterol levels. Such therapies include Hormone Replacement therapies, e.g., Premarin, raloxifene hydrochloride, available from Eli Lilly under the EVISTA tradename, as well as hypocholesterolemic agents such as ezetimibe disclosed in U.S. Pat. No. 5,767,115, and cholesterol biosynthesis inhibitors.

The term “cholesterol biosynthesis inhibitors” include 3-hydroxy-3-methylglutaryl coenzyme A (HMG CoA) reductase inhibitors, such as lovastatin, pravastatin, fluvastatin, itavastatin, simvastatin, ZD-4522 (available from AstraZeneca), and CI-981, as well as HMG CoA synthesis inhibitors, including for example, squalestatin 1, and squalene synthesis inhibitors, for example, NB-598 and other cholesterol biosynthesis inhibitors such as DMP-565. The preferred HMG CoA reductase inhibitors are lovastatin, itavastatin, simvastatin, and ZD-4522.

Treatment by administering loratadine and montelukast should be continued until there is improvement in the patient's condition. Lower immunoglobulin and/or eosinophil levels (compared to baseline levels) in the patients treated in accordance with the present invention indicates improvement in the patient's condition and risk for cardiovascular disease.

Improvement in the patients at risk may also be ascertained upon review of a complete physical and serological examination of the patient by an attending clinician.

While the invention has been described in conjunction with the specific embodiments set forth above, many alternatives, modifications and variations thereof will be apparent to those of ordinary skill in the art. All such alternatives, modifications and variations are intended to fall within the spirit and scope of the present invention.

We claim:

1. A method of treating and/or preventing a cardiovascular disease in a human suffering from an allergic and/or inflammatory condition which comprises administering to such human in need of such treating and/or preventing an effective amount of loratadine, or a pharmaceutically acceptable salt thereof, in combination with an effective amount of montelukast, or a pharmaceutically acceptable salt thereof.

2. The method of claim 1, wherein the effective amount of loratadine is about 1 mg/day to about 45 mg/day.

3. The method of claim 2, wherein the effective amount of loratadine is about 5 mg/day to about 15 mg/day.

4. The method of claim 3, wherein the effective amount of loratadine is about 5 mg/day to about 10 mg/day.

5. The method of claim 4, wherein the effective amount of loratadine is about 10 mg/day.

6. The method of claim 1, wherein the allergic and/or inflammatory is seasonal allergic rhinitis, perennial allergic rhinitis, atopic dermatitis, urticaria or allergic asthma.

7. The method of claim 1, wherein the effective amount of montelukast is about 5 mg/day to about 20 mg/day.

8. The method of claim 7, wherein the effective amount of montelukast is about 10 mg/day.

9. A method of treating and/or preventing a cardiovascular disease in a human in need of such treating and/or preventing which comprises administering to such human in need of such treating and/or preventing an effective amount of loratadine, or a pharmaceutically acceptable salt thereof, in combination with an effective amount of montelukast, or a pharmaceutically acceptable salt thereof.

10. The method of claim 9, wherein the effective amount of loratadine is in the range of about 1 mg/day to about 45 mg/day.

11. The method of claim 10, wherein the effective amount of loratadine is about 5 mg/day to about 15 mg/day.

12. The method of claim 11, wherein the effective amount of loratadine is about 5 mg/day to about 10 mg/day.

13. The method of claim 12, wherein the effective amount of loratadine is about 10 mg/day.

14. The method of claim 9, wherein the effective amount of montelukast is about 5 mg/day to about 20 mg/day.

15. The method of claim 14, wherein the effective amount of montelukast is about 10 mg/day.

16. A method of treating and/or preventing a cardiovascular disease in a human suffering from seasonal or perennial allergic rhinitis which comprises administering to such human in need of such treating and/or preventing an effective amount of loratadine, or a pharmaceutically acceptable salt thereof, in combination with an effective amount of montelukast, or a pharmaceutically acceptable salt thereof.

17. The method of claim 16, wherein the effective amount of loratadine is about 1 mg/day to about 45 mg/day.

18. The method of claim 17, wherein the effective amount of loratadine is about 5 mg/day to about 15 mg/day.

19. The method of claim 18, wherein the effective amount of loratadine is about 5 mg/day to about 10 mg/day.

20. The method of claim 19, wherein the effective amount of loratadine is about 10 mg/day.

21. The method of claim 16, wherein the human is suffering from seasonal allergic rhinitis.

22. The method of claim 16, wherein the human is suffering from perennial allergic rhinitis.

23. The method of claim 16, wherein the effective amount of montelukast is about 5 mg/day to about 20 mg/day.

24. The method of claim 23, wherein the effective amount of montelukast is about 10 mg/day.

25. A method of treating and/or preventing a cardiovascular disease in a human suffering from atopic dermatitis or urticaria which comprises administering to such human in need of such treating and/or preventing an effective amount of loratadine, or a pharmaceutically acceptable salt thereof, in combination with an effective amount of montelukast, or a pharmaceutically acceptable salt thereof.

26. The method of claim 25, wherein the effective amount of loratadine is about 1 mg/day to about 45 mg/day.

27. The method of claim 26, wherein the effective amount of loratadine is about 5 mg/day to about 15 mg/day.

28. The method of claim 27, wherein the effective amount of loratadine is about 5 mg/day to about 10 mg/day.

29. The method of claim 28, wherein the effective amount of loratadine is about 10 mg/day.

30. The method of claim 25, wherein the patient is suffering from atopic dermatitis.

31. The method of claim 25, wherein the patient is suffering from urticaria.

32. The method of claim 25, wherein the effective amount of montelukast is about 5 mg/day to about 20 mg/day.

33. The method of claim 32, wherein the effective amount of montelukast is about 10 mg/day.
